(19) United States
(12) Patent Application Publication
(10) Pub. No.: US 2026/0240848 A1
(43) Pub. Date: Aug. 20, 2026

(54) MYOSIN INHIBITORS FOR USE IN THE TREATMENT OF HYPERTROPHIC CARDIOMYOPATHY

(71) Applicant: Myokardia, Inc., Brisbane, CA (US)

(72) Inventors: Angelos OIKONOMOPOULOS, Brisbane, CA (US); Jay Edelberg, Brisbane, CA (US); Matthew Mealiffe, Brisbane, CA (US); Carlos Del Rio, Brisbane, CA (US)

(21) Appl. No.: 19/159,851

(22) PCT Filed: Feb. 28, 2024
(86) PCT No.: PCT/US2024/017590

§ 371 Date: Aug. 26, 2025

Related U.S. Application Data
(60) Provisional application No. 63/487,289, filed on Feb. 28, 2023.

(51) Int. Cl. A61K 31/513; A61K 31/4245; A61P 9/00; A61P 43/00

(52) U.S. Cl. CPC A61K 31/513; A61K 31/4245; A61P 9/00; A61P 43/00

(57) ABSTRACT

Methods of using myosin inhibitors for cytoprotection of cardiomyocytes from hypoxic injury or death are described herein. Studies described herein showed R403Q HCM cardiomyocytes were more susceptible to hypoxic injury compared to wild-type cardiomyocytes. In R403Q HCM cardiomyocytes under hypoxic conditions, hypoxia biomarker levels and cell nuclei counts trended towards normoxia levels upon treatment with the myosin inhibitor MYK-581.

### CROSS-REFERENCE TO RELATED APPLICATION

This application claims priority to and benefit of U.S. provisional patent application No. 63/487,289, filed Feb. 28, 2023, the entire contents of which are incorporated herein by reference.

### TECHNICAL FIELD

The present invention relates to the use of myosin inhibitors, e.g., MYK-581, mavacamten, or aficamten, for cytoprotection of cardiomyocytes from hypoxic injury or death.

### BACKGROUND

Hypertrophic cardiomyopathy (HCM) is a chronic, progressive disease in which excessive contraction of the heart muscle and reduced left ventricle filling capacity can lead to the development of debilitating symptoms, cardiac dysfunction, and sudden cardiac death. HCM is estimated to affect one in every 500 people. The most frequent cause of HCM is mutations in the proteins of the cardiac sarcomere. In approximately two-thirds of HCM subjects, the path followed by blood exiting the heart, known as the left ventricular outflow tract (LVOT), becomes obstructed by the enlarged and diseased muscle, restricting flow to the rest of the body (obstructive HCM). In other subjects, the thickened heart muscle does not block the LVOT, and their disease is driven by diastolic impairment due to the enlarged and stiffened heart muscle (non-obstructive HCM). In either obstructive or non-obstructive HCM subjects, exertion can result in fatigue or shortness of breath, interfering with a subject's ability to participate in activities of daily living. HCM has also been associated with increased risks of atrial fibrillation, stroke, heart failure and sudden cardiac death.

Troponin release is considered a marker of cardiac injury due to multiple types of injury including ischemia. Previous studies have revealed significant effects of myosin modulators on troponin release in cardiomyopathy patients. Treatment of heart failure patients with myosin activators (Omecamtiv) led to the detection of increased cardiac Troponin I (COSMIC-HF). On the contrary, myosin inhibitors (Mavacamten) inhibited the release of Troponin I in patients with oHCM (Explorer-HCM). Hypoxia is a pathogenic feature of hypertrophy due to the focal ischemic areas that are generated from the imbalance between the increased myocardial mass and reduced capillary density. Various cellular and molecular mechanisms have been implicated in the reduced cardiac angiogenesis during pathological hypertrophy. The present inventors investigated the effects of myosin inhibitors on Troponin release in wild-type and HCM mutant cardiomyocytes following hypoxic injury.

### SUMMARY

Provided herein is a method for prophylaxis of hypertrophic cardiomyopathy (HCM) or slowing the progression of HCM comprising administering to a patient in need thereof a therapeutically effective amount of a myosin inhibitor.

In some embodiments, the myosin inhibitor is MYK-581 or a pharmaceutically acceptable salt thereof. In some embodiments, the myosin inhibitor is MYK-581.

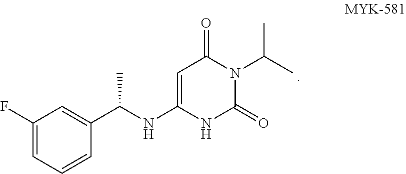 [SMILES: CC(C)n1c(=O)cc(N[C@@H](C)c2cccc(F)c2)[nH]c1=O]

In some embodiments, the myosin inhibitor is mavacamten or a pharmaceutically acceptable salt thereof. In some embodiments, the myosin inhibitor is mavacamten.

In some embodiments, they myosin inhibitor is aficamten or a pharmaceutically acceptable salt thereof. In some embodiments, the myosin inhibitor is aficamten.

In some embodiments, the method for prophylaxis or slowing the progression of HCM includes one or more the of the following optional features: the HCM is asymptomatic; the patient has a resting LVOT gradient of less than 50 mmHg; the patient has a resting LVOT gradient of less than 30 mmHg; the patient is not characterized as having diastolic heart failure; the patient is characterized as NYHA class I or II; the patient is characterized as NYHA class I; cardiac troponin T (cTnT) levels in the patient are decreased after administration of the myosin inhibitor; Fatty acid protein 3 (FABP3) levels in the patient are decreased after administration of the myosin inhibitor; and Myosin light chain 3 (Myl3) levels in the patient are decreased after administration of the myosin inhibitor.

Also provided herein is a method of reducing the oxygen demand of cardiomyocytes in a subject in need thereof comprising administering to the subject a therapeutically effective amount of a myosin inhibitor. In some embodiments, the subject has HCM. In some embodiments, the cardiomyocytes have a R403Q mutation.

Also provided herein is a method of preventing hypoxia-induced cell injury or death in cardiomyocytes in a subject in need thereof comprising administering to the subject a therapeutically effective amount of a myosin inhibitor. In some embodiments, the subject has HCM. In some embodiments, the cardiomyocytes have a R403Q mutation.

Also provided herein is a method of treating a disease selected from angina, ischemia, lung disease, or coronary heart disease by reducing hypoxia-induced cell injury or death, the method comprising administering to a patient in need thereof a therapeutically effective amount of a myosin inhibitor.

Also provided herein is a method of providing cytoprotection to an individual experiencing a reduced supply of oxygen (i.e. hypoxia), the method comprising administering to the individual an effective amount of a myosin inhibitor. In some embodiments, the reduced supply of oxygen is due to emphysema, COVID-19, space travel, exercise, heart attack or high-altitude sickness. In some embodiments, the individual experiences a reduced supply of oxygen available to cardiomyocytes.

In some embodiments, the method of (a) reducing the oxygen demand of cardiomyocytes, (b) preventing hypoxia-induced cell injury or death in cardiomyocytes, (c) treating a disease selected from angina, ischemia, lung disease, or coronary heart disease by reducing hypoxia-induced cell injury or death, or (d) providing cytoprotection to an individual experiencing a reduced supply of oxygen, includes one or more the of the following optional features: the myosin inhibitor is MYK-581 or a pharmaceutically acceptable salt thereof; the myosin inhibitor is mavacamten or a pharmaceutically acceptable salt thereof; the subject, individual, or patient has asymptomatic HCM; the subject, individual, or patient has a resting LVOT gradient of less than 50 mmHg; the subject, individual, or patient has a resting LVOT gradient of less than 30 mmHg; the subject, individual, or patient is not characterized as having diastolic heart failure; the subject, individual, or patient is characterized as NYHA class I or II; the subject, individual, or patient is characterized as NYHA class I; cardiac troponin T (cTnT) levels in the subject, individual, or patient are decreased after administration of the myosin inhibitor; Fatty acid protein 3 (FABP3) levels in the subject, individual, or patient are decreased after administration of the myosin inhibitor; Myosin light chain 3 (Myl3) levels in the subject, individual, or patient are decreased after administration of the myosin inhibitor; the cardiomyocytes are R403Q cardiomyocytes; the subject, individual, or patient has HCM.

### DRAWINGS

FIG. 1 is a flow chart of the study design of Example 1.

FIG. 2 is a flow chart of plate map/image acquisition and also shows the actual stained nuclei in the image, which was used for nuclei counting.

FIG. 3 shows biomarker results for WT and R403Q iPSC-CMs treated with MYK-581 for 16 hours.

FIG. 4 shows nuclei count results for WT and R403Q iPSC-CMs treated with MYK-581 for 16 hours.

FIG. 5 shows biomarker results for WT and R403Q iPSC-CMs treated with MYK-581 for 24 hours.

FIG. 6 shows nuclei count results for WT and R403Q iPSC-CMs treated with MYK-581 for 24 hours.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The terms “a” or “an,” as used in herein means one or more.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

The present inventors studied iPSC-derived cardiomyocytes (induced pluripotent stem cell-derived cardiomyocytes) and discovered greater rates of cell death under hypoxic conditions in HCM R403Q-mutant cardiomyocytes compared to isogenic control cells. The higher oxygen or energy need of HCM R403Q cells is consistent with the hypercontractility of HCM hearts observed clinically and in HCM myofibril studies. The increased oxygen consumption of HCM R403Q cells, in the absence of pressure gradient or diastolic failure in the cell culture, indicates an intrinsic property of HCM cardiomyocytes in high oxygen or energy demand. This intrinsic high demand of oxygen is on top of the clinical pressure gradient or diastolic heart failure, which further increases oxygen demand and impairs vascular oxygen supply to cardiomyocytes. Therefore, for asymptomatic HCM patients before the clinical development of pressure gradient or diastolic heart failure, treatments with myosin inhibitors may be used according to the present disclosure for cytoprotection of cardiomyocytes from hypoxic state.

In cell culture studies, the inventors found that myosin inhibitors (MYK-581) could reduce the oxygen demand of HCM R403Q cardiomyocytes in a dose-dependent manner to normalize the oxygen consumption to the isogenic control level. In addition, the high oxygen demand of HCM cardiomyocytes predisposed those cells to hypoxia-induced cell death, which was ameliorated by treating those cells with myosin inhibitors, consistent with the energy-sparing effects of myosin inhibitors. Overall, the data support the utility of myosin inhibitors in energy sparing and protection of HCM cardiomyocytes against hypoxia or ischemic insult that is common in HCM patients. A detailed description of the cell culture studies and results is provided herein in Example 1.

The hypoxic state of HCM R403Q-mutant cardiomyocytes has been observed through assays of cell death and in certain biomarkers correlating to cell death. Cell viability can be measured with a Cell Titer Glo kit. Biomarkers correlating to cell death in HCM cardiomyocytes include cardiac troponin T (cTnT), Fatty acid protein 3 (FABP3), and Myosin light chain 3 (Myl3). Nuclei counts using imaging can also be used as a measure of hypoxic cell death/survival.

The present invention relates to the use of myosin inhibitors for cytoprotection of cardiomyocytes in HCM patients from hypoxic injury or death.

In some embodiments, a myosin inhibitor is a compound of formula (I):

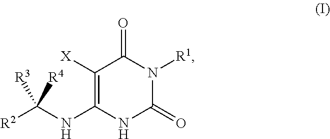

or pharmaceutically acceptable salt thereof, wherein: R¹ is C₁₋₈ alkyl, C₃₋₈ cycloalkyl, or a phenyl, wherein R¹ is optionally substituted with one or two halo;
R² is phenyl optionally substituted with one or two halo;
R³ is C₁₋₈ alkyl or C₃₋₈ cycloalkyl, wherein R³ is optionally substituted with halo, hydroxyl or C₁₋₂ alkoxy;
R⁴ is H; and
X is H.

In some embodiments, a myosin inhibitor of formula (I) or a pharmaceutically acceptable salt thereof is selected from group (I) consisting of:

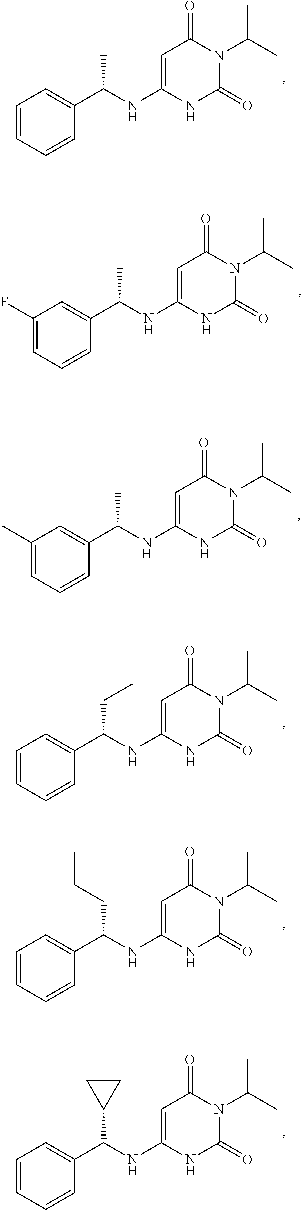

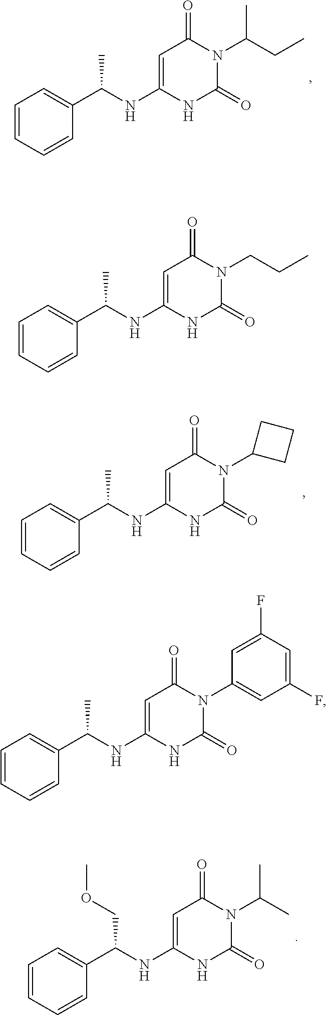 [SMILES: CCC(C)n1c(=O)cc(N[C@@H](C)c2ccccc2)[nH]c1=O ; CCCn1c(=O)cc(N[C@@H](C)c2ccccc2)[nH]c1=O ; C[C@H](Nc1cc(=O)n(C2CCC2)c(=O)[nH]1)c1ccccc1 ; C[C@H](Nc1cc(=O)n(-c2cc(F)cc(F)c2)c(=O)[nH]1)c1ccccc1 ; COC[C@H](Nc1cc(=O)n(C(C)C)c(=O)[nH]1)c1ccccc1]

In some embodiments, a myosin inhibitor of formula (I) is mavacamten or a pharmaceutically acceptable salt thereof. In some embodiments, a myosin inhibitor of formula (I) is mavacamten. Mavacamten has the following structure:

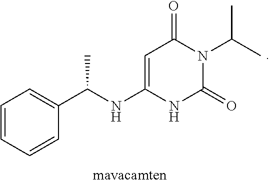 [SMILES: CC(C)n1c(=O)cc(N[C@@H](C)c2ccccc2)[nH]c1=O]

Mavacamten is also known as MYK-461. Its chemical name is(S)-3-Isopropyl-6-((1-phenylethyl)amino)pyrimidine-2,4 (1H,3H)-dione or 6-[[(1S)-1-phenylethyl]amino]-3-propan-2-yl-1H-pyrimidine-2,4-dione.

In some embodiments, a myosin inhibitor of formula (I) is MYK-581 or a pharmaceutically acceptable salt thereof. In some embodiments, a myosin inhibitor of formula (I) is MYK-581. MYK-581 has the following structure.

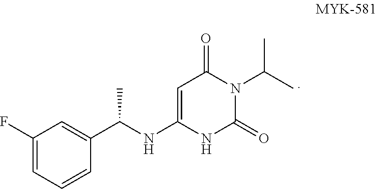 [SMILES: CC(C)n1c(=O)cc(N[C@@H](C)c2cccc(F)c2)[nH]c1=O]

MYK-581's chemical name is(S)-6-((1-(3-fluorophenyl)ethyl)amino)-3-isopropylpyrimidine-2,4 (1H,3H)-dione.

Myosin inhibitors of formula (I), including the compounds of group (I), mavacamten, or MYK-581, or a pharmaceutically acceptable salt thereof, can be obtained according to the production methods described in U.S. Pat. No. 9,181,200, which is incorporated herein by reference in its entirety and for all purposes.

In some embodiments, the myosin inhibitor is aficamten or a pharmaceutically acceptable salt thereof. Aficamten has the following structure:

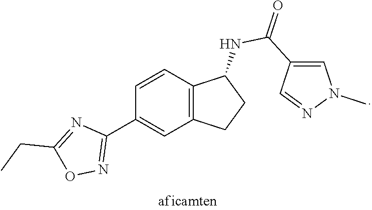 [SMILES: CCc1nc(-c2ccc3c(c2)CC[C@H]3NC(=O)c2cnn(C)c2)no1]

In some embodiments, a compound of formula (I), and/or a compound of group (I), and/or mavacamten, and/or MYK-581, and/or aficamten is administered orally.

In some embodiments, a compound of formula (I), and/or a compound of group (I), and/or mavacamten, and/or MYK-581, and/or aficamten is administered in a unit dosage.

In some embodiments, mavacamten, and/or MYK-581, and/or aficamten is administered at a daily dosage amount of 1 mg, 2 mg, 2.5 mg, 5 mg, 7.5 mg, 10 mg, or 15 mg.

In some embodiments, mavacamten, and/or MYK-581, and/or aficamten is administered daily for 4 weeks, 8 week, 12 weeks, 18 weeks, 24 weeks, 30 weeks, 36 weeks, 48 weeks, or 56 weeks at a daily dosage amount of 1 mg, 2 mg, 2.5 mg, 5 mg, 7.5 mg, 10 mg, or 15 mg.

In some embodiments, mavacamten, and/or MYK-581, and/or aficamten is administered daily at a starting treatment dosage of 2.5 mg per day and optionally increased to 5 mg per day if certain conditions are met.

In some embodiments, mavacamten, and/or MYK-581, and/or aficamten is chronically administered daily at least one year, two year, three year, more than five year, or as long as determined by a physician, at a daily dosage amount of 1 mg, 2 mg, 2.5 mg, 5 mg, 7.5 mg, 10 mg, or 15 mg as a maintenance therapy.

The pharmaceutical compositions for the administration of a compound of formula (I), and/or a compound of group (I), and/or mavacamten, and/or MYK-581, and/or aficamten or a pharmaceutically acceptable salt thereof may conveniently be presented in unit dosage form and may be prepared by any of the methods known in the art of pharmacy and drug delivery. All methods include the step of bringing the active ingredient into association with a carrier containing one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active agent is generally included in an amount sufficient to produce the desired effect upon myocardial contractility (i.e. to decrease the often supranormal systolic contractility in HCM) and to improve left ventricular relaxation in diastole. Such improved relaxation can alleviate symptoms in hypertrophic cardiomyopathy and other etiologies of diastolic dysfunction. It can also ameliorate the effects of diastolic dysfunction causing impairment of coronary blood flow, improving the latter as an adjunctive agent in angina pectoris and ischemic heart disease. It can also confer benefits on adverse left ventricular remodeling in HCM and other causes of left ventricular hypertrophy due to chronic volume or pressure overload from, e.g., valvular heart disease or systemic hypertension.

The pharmaceutical compositions containing a compound of formula (I), and/or a compound of group (I), and/or mavacamten, and/or MYK-581, and/or aficamten or a pharmaceutically acceptable salt thereof, may be in a form suitable for oral use, for example, as tablets, troches, lozenges, aqueous or oily suspensions, dispersible powders or granules, emulsions, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

In some embodiments, a compound of formula (I), and/or a compound of group (I), and/or mavacamten, and/or MYK-581, and/or aficamten can be used in the form of pharmaceutically acceptable salt. Examples of the pharmaceutically acceptable salt include salts with inorganic bases, salts with organic bases, salts with inorganic acids, salts with organic acids, and salts with basic or acidic amino acids.

Myosin inhibitors, including a compound of formula (I), and/or a compound of group (I), and/or mavacamten, and/or MYK-581, and/or aficamten and compositions thereof are useful in the methods disclosed herein.

Provided herein is a method for prophylaxis of hypertrophic cardiomyopathy (HCM) or slowing the progression of HCM comprising administering to a patient in need thereof a therapeutically effective amount of a myosin inhibitor. The cytoprotective effect of myosin inhibitors on cardiomyocytes, demonstrated by the study and results described herein, is useful for preventing hypoxic injury or death to cardiomyocytes, which in turn is useful for prophylaxis of or slowing the progression of HCM.

In some embodiments of the method of prophylaxis of or slowing the progression of HCM, the HCM is asymptomatic. The study and results described herein showed a greater cytoprotective effect when cells were treated with a myosin inhibitor in the early stages of hypoxia. Treatment during early stages of HCM, while HCM is asymptomatic, is therefore contemplated herein. Other characteristics consistent with early stages of HCM are also contemplated herein. In some embodiments, the patient has a resting LVOT gradient of less than 50 mmHg or less than 30 mmHg. In some embodiments, the patient is not characterized as having diastolic heart failure. In some embodiments, the patient is characterized as NYHA class I or II, e.g. NYHA class I. In some embodiments, the patient has vascular dysplasia, which can be detected by PET scan.

In some embodiments, cardiac troponin T (cTnT) levels, Fatty acid protein 3 (FABP3) levels, and/or Myosin light chain 3 (Myl3) levels in the patient are decreased after administration of the myosin inhibitor. In some embodiments, cardiac troponin T (cTnT) levels, Fatty acid protein 3 (FABP3) levels, and/or Myosin light chain 3 (Myl3) levels are maintained at or near normoxia levels after administration of the myosin inhibitor.

The NYHA functional classification grades the severity of heart failure symptoms as one of four functional classes. The NYHA functional classification is widely used in clinical practice and in research because it provides a standard description of severity that can be used to assess response to treatment and to guide management. The NYHA functional classification based on severity of symptoms and physical activity are:
Class I: No limitation of physical activity. Ordinary physical activity does not cause undue breathlessness, fatigue, or palpitations
Class II: Slight limitation of physical activity. Comfortable at rest, but ordinary physical activity results in undue breathlessness, fatigue, or palpitations.
Class III: Marked limitation of physical activity. Comfortable at rest, but less than ordinary physical activity results in undue breathlessness, fatigue, or palpitations.
Class IV: Unable to carry on any physical activity without discomfort. Symptoms at rest can be present. If any physical activity is undertaken, discomfort is increased.

Also provided herein is a method of reducing the oxygen demand of cardiomyocytes in a subject in need thereof comprising administering to the subject a therapeutically effective amount of a myosin inhibitor. Survival of cardiomyocytes (e.g., as measured by nuclei counts) upon exposure to hypoxic conditions indicates a reduced oxygen demand. In some embodiments, the cardiomyocytes have a R403Q mutation. In some embodiments, the subject has HCM.

Also provided herein is a method of preventing hypoxia-induced cell injury or death in cardiomyocytes in a subject in need thereof comprising administering to the subject a therapeutically effective amount of a myosin inhibitor. In some embodiments, the cardiomyocytes have a R403Q mutation. In some embodiments, the subject has HCM.

Also provided herein is a method of treating a disease selected from angina, ischemia, lung disease, or coronary heart disease by reducing hypoxia-induced cell injury or death, the method comprising administering to a patient in need thereof a therapeutically effective amount of a myosin inhibitor.

Also provided herein is a method of providing hypoxic cytoprotection to an individual experiencing a reduced supply of oxygen, the method comprising administering to the individual an effective amount of a myosin inhibitor. In some embodiments, the reduced supply of oxygen is due to emphysema, COVID-19, space travel, exercise, heart attack, or high altitude sickness. In some embodiments, the individual experiences a reduced supply of oxygen available to cardiomyocytes. Increased oxygen demand and decreased oxygen supply in COVID-19 patients may lead to heart damage, which may be treated or prevented by cytoprotection according to the methods of the present invention. So-called cytokine storm events in COVID-19 patients may also lead to heart damage, which may be treated or prevented by cytoprotection according to the methods of the present invention.

In some embodiments of the methods described herein, the HCM is non-obstructive HCM (nHCM); in other embodiments, the HCM is obstructive HCM (oHCM).

### EXAMPLES

### Example 1

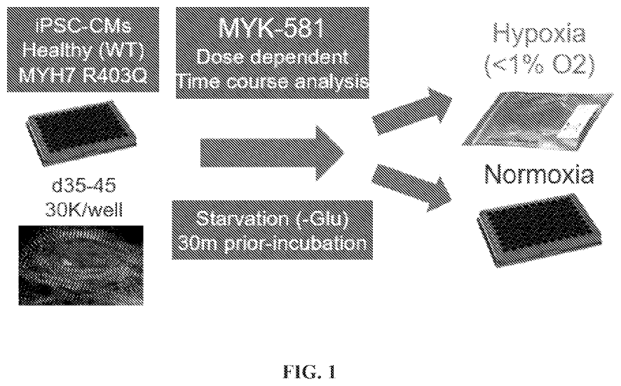

Cell lines were obtained for Wild Type (WT) and R403Q HCM cardiomyocytes. The cell lines were derived from the same genetic background (isogenic lines). The cells were switched to medium containing no glucose and were treated with MYK-581 (or control) in a dose dependent manner prior to hypoxic injury, and using normoxic conditions (i.e., normal atmospheric oxygen conditions) as a control. Hypoxic injury was induced by exposing the cells to an environment with less than 1% oxygen, i.e., anoxic conditions. A flow chart of the study design is shown in FIG. 1.

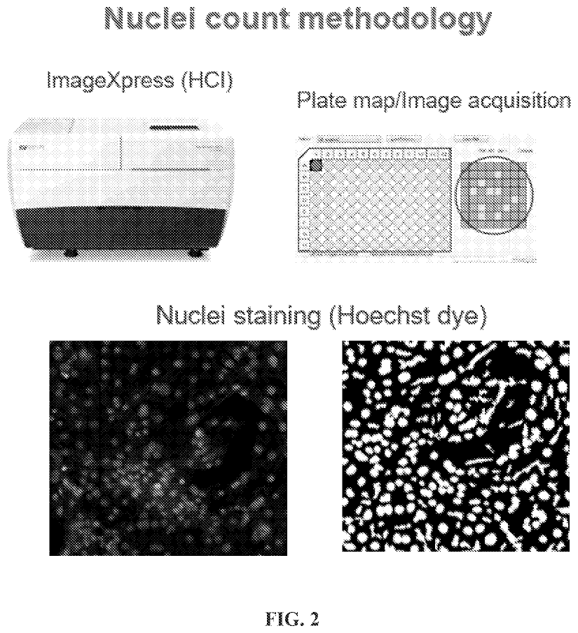

Cells were analyzed for viability using a Cell Titer Glo kit (ATP content-CTG). Nuclei counts were also measured. For measuring the nuclei count, at the end of each experiment, each plate was stained with Hoechst 33442, a DNA binding dye, and was imaged in the ImageXpress (Molecular Devices, LLC, San Jose, CA). Plate map/image acquisition was performed as shown in FIG. 2. FIG. 2 also shows the actual stained nuclei in the image after processing by the ImageXpress, which was used for nuclei counting.

A Meso Scale discovery (MSD) platform was used for biomarker measurements. The MSD platform for biomarker detection was applied in cell supernatant, which is the conditioned cell media (external to the cell layer). The MSD platform functions similar to a traditional ELISA, with the distinction that a voltage is applied across an electrode at the bottom of each well such that an electrochemiluminescent signal is produced. For this study, the Muscle Injury Panel 1 Rat Kit (Cat #K15181C, Meso Scale Diagnostics) was used, which takes advantage of multiplexing. Five analytes from a single sample were measured in one experiment-cardiac troponin I (cTnI), cardiac troponin T (cInT), fatty acid binding protein 3 (FABP3), myosin light chain 3 (Myl3), and skeletal troponin I (sTnI). Because this kit is intended for rat samples, cross-reactivity with the human form of the biomarker proteins was verified to ensure accurate relative quantification with iPSC-CMs (Induced pluripotent stem-cell derived cardiomyocytes).

Cell supernatants from iPSC-CM experiments were stored at −80° C. and thawed on ice prior to biomarker analysis. Cell supernatant sample were analyzed using supplied reagents and according to the manufacturer's protocol; plates were read on the MESO QuickPlex SQ 120 (Meso Scale Diagnostics). Relative biomarker quantification was based on a custom blend of protein standards: human cardiac troponin T (Cat #8-TTc-h, Advanced ImmunoChemical), human fatty acid binding protein 3 (Cat #8-FABP, Advanced ImmunoChemical), and human myosin light chain 3 (Cat #MBS957204, MyBioSource). The Muscle Injury Panel 1 Rat Kit was able to reproducibly detect the following biomarkers in cell supernatant: cardiac troponin T, fatty acid binding protein 3, and myosin light chain 3.

### Results

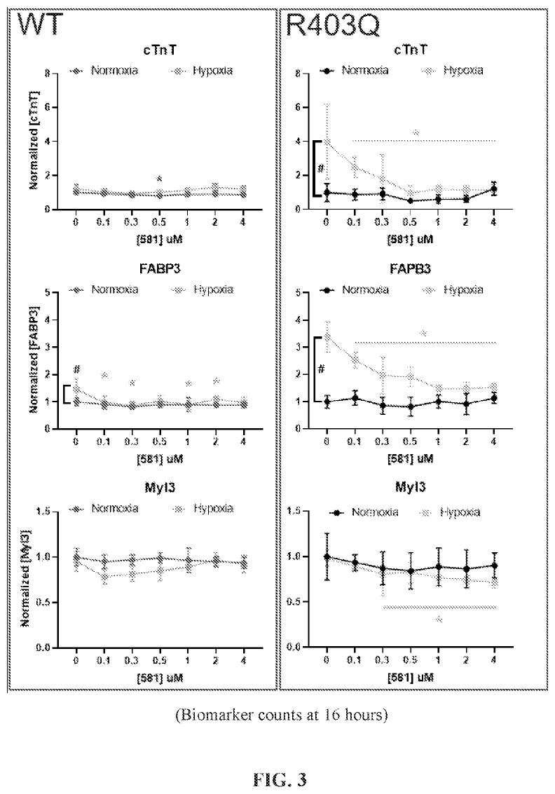

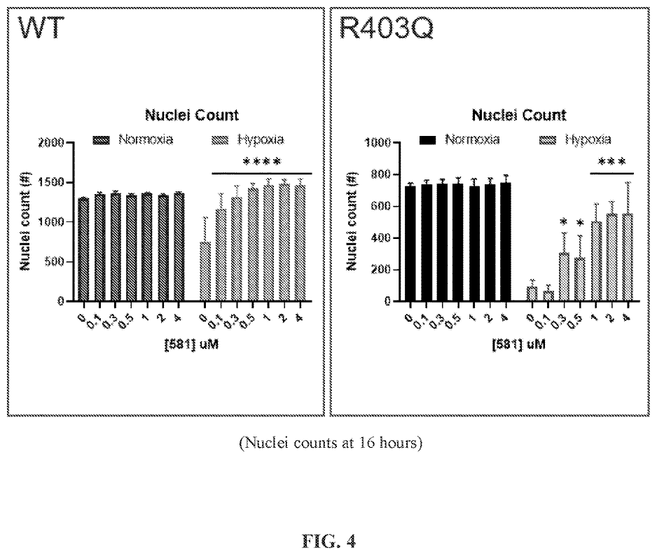

Biomarker and nuclei count results for WT and R403Q iPSC-CMs treated with MYK-581 for 16 hours are shown in FIGS. 3 and 4.

Due to differences in cell seeding densities between the cell lines, the data is normalized to normoxia, 0 μM MYK-581 for each cell line (WT vs R403Q). Statistical significance between normoxia and hypoxia at 0 μM MYK-581 is indicated by the #symbol. Statistical significance between MYK-581 treated cells and 0 μM MYK-581 cells within each condition (normoxia or hypoxia) is indicated by a color-coded*symbol.

cTnT: There are no major differences in the level of cInT in wild type (WT) cell supernatant between normoxia/hypoxia and with/without MYK-581 treatment. However, in R403Q cells not treated with MYK-581, cInT in the supernatant is elevated in hypoxia as compared to normoxia. The level of this biomarker in hypoxia decreases with increasing concentration of MYK-581, and is similar to levels observed in normoxia at 1-4 μM MYK-581.

FABP3: With no MYK-581 treatment, WT cells show an elevated amount of FABP3 in the cell supernatant in hypoxia as compared to normoxia; for WT cells treated with MYK-581, FABP3 levels are similar between normoxia and hypoxia. For R403Q cells not treated with MYK-581, FABP3 in the supernatant is elevated in hypoxia as compared to normoxia. The level of this biomarker in hypoxia decreases with increasing concentration of MYK-581 and is close to levels in normoxia at 1-4 μM MYK-581.

Myl3: There are no major differences in the level of Myl3 in wild type (WT) cell supernatant between normoxia/hypoxia and with/without MYK-581 treatment. In R403Q cells in hypoxia, there is a slight but statistically significant decrease in the level of this biomarker with MYK-581 treatment (0.3-4 μM).

MYK-581 significantly decreases the levels of the stress biomarkers in the supernatants of the cells. Nuclei counting of WT cells in normoxic conditions revealed no difference across the plate. However, in hypoxic conditions, vehicle treated cells had significantly lower nuclei count compared to MYK-581-treated wells. This effect was mediated at low concentrations of the drug at 0.1 μM.

Similarly, when measuring the cell nuclei in the R403Q cells, there was no difference in the normoxic conditions. However, in hypoxic conditions, there was a profound decrease in nuclei count that was reversed by the addition of MYK-581, starting at 0.3 μM.

Overall, R403Q cells are more susceptible to hypoxic injury, confirming Cell Titer Glo results. MYK-581 mediates protective effects in hypoxic conditions at low concentrations.

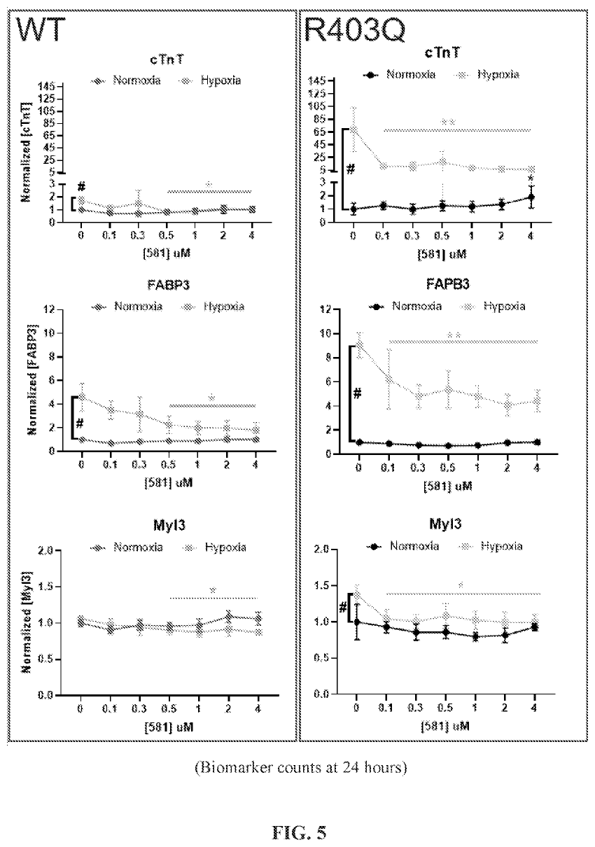

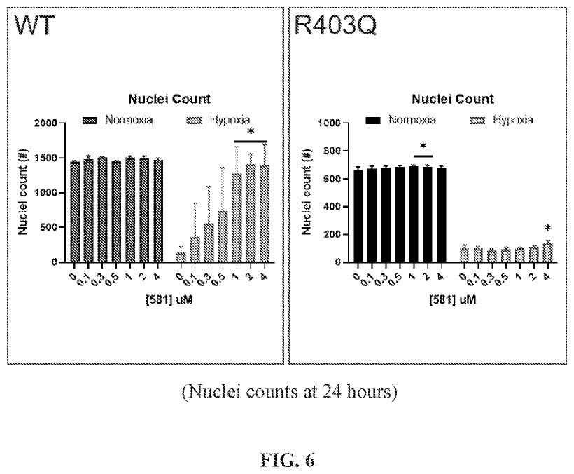

Biomarker and nuclei counts were also measured for WT and R403Q iPSC-CMs treated with MYK-581 for 24 hours. Results are shown in FIGS. 5 and 6.

cTnT: With no MYK-581 treatment, WT cells show an elevated amount of cTnT in the cell supernatant in hypoxia as compared to normoxia; for WT cells treated with MYK-581, cTnT levels are similar between normoxia and hypoxia. This is in contrast with the 16 hr data, in which no statistically significant difference was observed in this biomarker between normoxia and hypoxia for WT cells without MYK-581 treatment. For R403Q cells not treated with MYK-581, cTnT in the supernatant is elevated in hypoxia as compared to normoxia, and to a greater degree than at 16 hrs. The level of this biomarker in hypoxia decreases with MYK-581 treatment; however, in contrast to the 16 hr data, levels remain elevated relative to normoxia up to 4 μM MYK-581.

FABP3: WT cells show an elevated amount of FABP3 in the cell supernatant in hypoxia as compared to normoxia; the level of this biomarker in hypoxia decreases with increasing concentration of MYK-581. For R403Q cells not treated with MYK-581, FABP3 in the supernatant is elevated in hypoxia as compared to normoxia, and to a greater degree than at 16 hrs. The level of this biomarker in hypoxia decreases with increasing concentration of MYK-581; however, in contrast to the 16 hr data, levels remain elevated relative to normoxia up to 4 μM MYK-581.

Myl3: For WT cells, there is no major difference in the level of this biomarker between normoxia and hypoxia. There is a slight but statistically significant decrease in the level of this biomarker for WT cells in hypoxia with MYK-581 treatment (0.5-4 μM). With no MYK-581 treatment, R403Q cells show an elevated amount of Myl3 in the cell supernatant in hypoxia as compared to normoxia, and the level of this biomarker decreases in hypoxia with MYK-581 treatment.

At this time point (24 h), the injury is much more severe for both cell lines. In the WT cells, there are much fewer nuclei in vehicle treated sample but this was reversed by MYK-581 treatment. In the R403Q cells, very few nuclei were detected in both vehicle and MYK-581 treated samples showing again that the R403Q cells are more sensitive to the injury compared to WT cells at this time point (24 hr).

### CONCLUSIONS

R403Q iPSC-CMs are more susceptible to hypoxia injury compared to WT cells. Injury is more pronounced with longer duration of hypoxic conditions. Hypoxia biomarker levels and cell nuclei counts trended towards normoxia levels with compound treatment, which suggests a cytoprotective effect of MYK-581. Cytoprotection by MYK-581 is observed at concentrations near the IC50 (~0.2 μM in human iPSC-CMs).

We claim:

1. A method for prophylaxis of hypertrophic cardiomyopathy (HCM) or slowing the progression of HCM comprising administering to a patient in need thereof a therapeutically effective amount of a myosin inhibitor.

2. The method of claim 1, wherein the myosin inhibitor is MYK-581 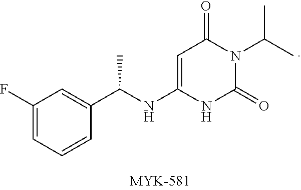 [SMILES: CC(C)n1c(=O)cc(N[C@@H](C)c2cccc(F)c2)[nH]c1=O]
or a pharmaceutically acceptable salt thereof.

3. The method of claim 2, wherein the myosin inhibitor is MYK-581.

4. The method of claim 1, wherein the myosin inhibitor is mavacamten or aficamten, or a pharmaceutically acceptable salt thereof.

5. The method of claim 4, wherein the myosin inhibitor is mavacamten.

6. The method of any one of claims 1-5, wherein the HCM is asymptomatic.

7. The method of any one of claims 1-6, wherein the patient has a resting LVOT gradient of less than 50 mmHg.

8. The method of claim 7, wherein the patient has a resting LVOT gradient of less than 30 mmHg.

9. The method of any one of claims 1-8, wherein the patient is not characterized as having diastolic heart failure.

10. The method of any one of claims 1-9, wherein the patient is characterized as NYHA class I or II.

11. The method of claim 10, wherein the patient is characterized as NYHA class I.

12. The method of any one of claims 1-11, wherein cardiac troponin T (cTnT) levels in the patient are decreased after administration of the myosin inhibitor.

13. The method of any one of claims 1-12, wherein Fatty acid protein 3 (FABP3) levels in the patient are decreased after administration of the myosin inhibitor.

14. The method of any one of claims 1-13, wherein Myosin light chain 3 (Myl3) levels in the patient are decreased after administration of the myosin inhibitor.

15. A method of reducing the oxygen demand of cardiomyocytes in a subject in need thereof comprising administering to the subject a therapeutically effective amount of a myosin inhibitor.

16. A method of preventing hypoxia-induced cell injury or death in cardiomyocytes in a subject in need thereof comprising administering to the subject a therapeutically effective amount of a myosin inhibitor.

17. A method of treating a disease selected from angina, ischemia, lung disease, or coronary heart disease by reducing hypoxia-induced cell injury or death, the method comprising administering to a patient in need thereof a therapeutically effective amount of a myosin inhibitor.

18. A method of providing hypoxic cytoprotection to an individual experiencing a reduced supply of oxygen, the method comprising administering to the individual an effective amount of a myosin inhibitor.

19. The method of claim 18, wherein the reduced supply of oxygen is due to emphysema, COVID-19, space travel, exercise, heart attack, or high altitude sickness.

20. The method of claim 18 or 19, wherein the individual experiences a reduced supply of oxygen available to cardiomyocytes.

21. The method of any one of claims 15-20, wherein the myosin inhibitor is MYK-581 or a pharmaceutically acceptable salt thereof.

22. The method of claim 21, wherein the myosin inhibitor is MYK-581.

23. The method of any one of claims 15-20, wherein the myosin inhibitor is mavacamten or aficamten, or a pharmaceutically acceptable salt thereof.

24. The method of claim 23, wherein the myosin inhibitor is mavacamten.

25. The method of any one of claims 15-24, wherein the subject, individual, or patient has asymptomatic HCM.

26. The method of any one of claims 15-25, wherein the subject, individual, or patient has a resting LVOT gradient of less than 50 mmHg.

27. The method of claim 26, wherein the subject, individual, or patient has a resting LVOT gradient of less than 30 mmHg.

28. The method of any one of claims 15-27, wherein the subject, individual, or patient is not characterized as having diastolic heart failure.

29. The method of any one of claims 15-28, wherein the subject, individual, or patient is characterized as NYHA class I or II.

30. The method of claim 29, wherein the subject, individual, or patient is characterized as NYHA class I.

31. The method of any one of claims 15-30, wherein cardiac troponin T (cTnT) levels in the subject, individual, or patient are decreased after administration of the myosin inhibitor.

32. The method of any one of claims 15-31, wherein Fatty acid protein 3 (FABP3) levels in the subject, individual, or patient are decreased after administration of the myosin inhibitor.

33. The method of any one of claims 15-32, wherein Myosin light chain 3 (Myl3) levels in the subject, individual, or patient are decreased after administration of the myosin inhibitor.

34. The method of claim 15 or 16, wherein the cardiomyocytes have a R403Q mutation.

35. The method of any one of claims 15-34, wherein the subject, individual, or patient has HCM.
